(19) United States
(12) Patent Application Publication
(10) Pub. No.: US 2021/0307892 A1
(43) Pub. Date: Oct. 7, 2021

(54) CAROTID STENT INCORPORATING ARCH FULCRUM CATHETERS AND FLOW REVERSAL

(71) Applicant: Daniel Ezra Walzman, Bergenfield, NJ (US)

(72) Inventor: Daniel Ezra Walzman, Bergenfield, NJ (US)

(21) Appl. No.: 17/279,210

(22) PCT Filed: Sep. 14, 2020
(86) PCT No.: PCT/US20/50617

§ 371 Date: May 14, 2021

Related U.S. Application Data
(63) Continuation of application No. 16/575,302, filed on Sep. 18, 2019.

(51) Int. Cl. A61F 2/01; A61M 25/00; A61B 5/00; A61F 2/856; A61M 25/10; A61F 2/954

(52) U.S. Cl. CPC A61F 2/014; A61M 25/0041; A61B 5/6852; A61F 2/856; A61F 2002/9528; A61M 25/10; A61F 2/954; A61M 2210/12; A61M 25/0052

(57) ABSTRACT

A medical device for treating a vascular narrowing within a blood vessel including a catheter having a proximal end hole, a distal end hole positioned opposite the proximal end hole, a circumferential balloon located proximally of the distal end hole and an operational lumen extending through the catheter from the proximal end hole to the distal end hole. A first bend curves in a first direction and a second bend curves in a second generally opposite direction, the second bend being positioned distally of the first bend and proximally of the circumferential balloon, wherein the first bend and the second bend are configured to brace the catheter within the blood vessel against an arch defined by the blood vessel to inhibit recoil of the catheter. A supplemental medical device is configured for insertion into the blood vessel through the operational lumen of the catheter.

### FIELD OF THE INVENTION

This invention relates generally to endovascular devices and more particularly to a specifically shaped support catheter which provides a system for embolic protection. More particularly, the present invention is directed to a device that can obviate the need for an open surgical cutdown of the common carotid artery (CCA) with a carotid stent, using a flow reversal loop system for embolic protection, while also employing a percutaneous technique and novel carotid access devices which use anatomical fulcrums and/or unique steering capabilities for added support.

### BACKGROUND OF THE INVENTION

Minimally invasive treatments are increasingly popular including intravascular catheter treatments. Said treatments may be more effective than the prior art, nonetheless, in some embodiments they require an open surgical cutdown of the carotid artery which requires more anesthetic to perform typically than percutaneous procedures, with attendant anesthetic risks. Additionally, said procedures require surgical expertise, and presents additional risks of surgical injuries and/or infection at the cutdown site in the neck.

The present disclosure relates to methods and systems for accessing the carotid arterial vasculature and establishing retrograde blood flow during performance of carotid artery stenting and other procedures.

Carotid artery disease commonly results in deposits of plaque which narrow the junction between the common carotid artery CCA and the internal carotid artery ICA, an artery which provides blood flow to the brain. Said deposits may result in embolic particles being generated and entering the cerebral vasculature, leading to neurologic consequences such as transient ischemic attacks TIA, ischemic stroke, or death.

Various therapies exist to ameliorate carotid artery disease related difficulties. The most common are carotid endarterectomy CEA and a carotid artery stenting CAS. Both expose patients to the risk of emboli being released into the cerebral vasculature via the internal carotid artery ICA.

In response, recent prior art discloses a variety of devices and associated methods. For example, methods comprise trans-cervical access and blocking of blood flow through the common carotid artery while shifting blood from the internal carotid (see U.S. Ser. No. 12/835,660, U.S. Ser. No. 10/996,301, Ser. No. 12/366,287, Ser. No. 12/366,287 and Ser. No. 15/044,493).

The prior art discloses trans-carotid arterial revascularization. In particular, a small incision is made just above the collar bone and surgical dissection is used to surgically expose the common carotid artery. A soft, flexible tube (sheath) is placed directly into the carotid artery, and a clamp is applied to the external surface of the common carotid artery around the tube (sheath), and the tube (sheath) is connected to a system that will reverse the flow of blood away from the brain to protect against fragments of plaque that may come loose during the procedure. The blood is filtered and returned through a second tube (sheath) placed in the femoral vein in the patient's thigh or another vessel. Thereupon, the prior art also discloses balloon angioplasty and stenting performed while blood flow is reversed, and after the stent is placed successfully to stabilize the plaque in the carotid artery, the clamp is released and flow reversal is turned off and blood flow to the brain resumes in its normal direction. However, the prior art requires a surgical cut-down and dissection of the common carotid artery in the neck. Said surgery tends to disfigure the patient, requires additional anesthesia, additional training, and has a risk of damaging nerves.

Thus, there is a need for direct surgical access because of difficulties encountered with endovascular access, which can make adequate access difficult and higher risk in many cases.

There is a need for a less invasive percutaneous procedure, with lower risks of disfigurement, a lower risk of nerve injury, less use of anesthesia, and a simpler system requiring less training, thereby improving access to such treatments. The present invention addresses these unmet needs in the prior art.

The prior art discloses a set of Walzman arch fulcrum catheters, for example U.S. patent application Ser. No. 15/932,775 and Ser. No. 16/290,923, which may be useful to overcome this difficulty. In particular, a version with a balloon on a distal end may be used. Using this device, a user may consistently obtain transfemoral carotid access with adequate support with little difficulties and lower risks, and achieve similar results via a percutaneous transfemoral access, with less needs for anesthetics and their attendant risks.

Additionally, the prior art discloses a set of Walzman radial access catheters, which can make safe percutaneous access of either carotid artery feasible in the vast majority of patients, for example Ser. No. 16/501,591. Said catheters may also reduce access-site complications further. Said catheters can occasionally herein be further modified with at least one additional lumen substantially in the wall of said catheter, that can exit the wall of said catheter via at least one perforation in the outer wall of said catheter, to provide irrigation proximal to the balloon when said balloon is inflated, so as to minimize formation of clot proximal to said balloon. Such clots can form when a balloon occludes a vessel and causes stasis of blood.

### SUMMARY OF THE INVENTION

The present invention combines minimally invasive percutaneous endovascular carotid-artery access with rigorous blood flow-reversal, in order to protect the brain from embolic debris when introducing interventional devices into the carotid artery. In particular, the present invention uses reverse flow elements to prevent flow of blood to the brain, thus allowing maximal medical devices to be delivered to target areas more safely. Said reverse flow techniques include vaso-plugs, pumps, and irrigation distal to a lesion, among others.

The invention may be embodied in the form of either of two preferred devices—one for right carotid stenosis and one for left carotid stenosis. The current invention includes a (main, delivery) catheter (which may include one or more inflatable balloons mounted to an outer surface thereof) that is optimized for percutaneous access of the right and left carotid arteries in which a portion of the catheter is optimized to rest upon the lesser curvature of the aortic arch, in order to increase support for the delivery of additional medical devices (e.g., catheters, hypotubes, balloons, stents, etc.) through said (main, delivery) catheter, while also preventing recoil and kickback of said (main, delivery) catheter and devices inserted therethrough into the aortic arch, thereby improving procedural efficacy and reducing procedural risks.

The current invention, in other embodiments, may use transfemoral percutaneous endovascular access via additional arch fulcrum access catheters cross-referenced hereinabove, and additionally may use any of the previously described radial access catheters described herein. Additional embodiments may use alternatively various catheters described in the prior art, along with novel devices and methods to increase flow reversal at the lesion site, while minimizing the necessary diameter of the delivery catheter, and while minimizing any potential sump effect from the brain.

In still another embodiment of the current invention, a hollow wire is employed. The advantages of using a hollow wire include the ability to infuse fluids through it. This can

A. Decrease sump effect during flow reversal to minimize distal tissue ischemia;

B. Infuse blood if there is tissue ischemia;

C. Infuse neuroprotective solutions, such as cooled fluids; and

D. Infuse other material.

The benefits of using pressurized and/or pumped infusions is the ability to deliver higher rates (volume/time) of fluid through a small diameter lumen, thereby keeping the diameter of the devices as small as possible. This decreases risks at treatment site and access site, and increases range if access sites available (especially making treatment via radial artery access in most patients).

Additionally, using these methods and devices for carotid access can improve ease of percutaneous carotid access in many of the most difficult anatomical scenarios, thereby decreasing the risks of these percutaneous approaches. Catheters according to the present disclosure that are optimized for right carotid access via a transfemoral route will typically have a longer segment resting on the lesser arch of the aorta than corresponding catheter that are optimized for left carotid access. Embodiments include transfemoral and arm access arch fulcrum catheters. All catheters may optionally have active steerability of their respective bends. As is known in the art, this may sometimes be accomplished by the presence of wires in the wall of the catheter, with a mechanism to shorten the effective length of a wire to create a bend.

In one aspect of the disclosure, a medical device is disclosed for treating a vascular narrowing within a blood vessel. The medical device includes a catheter and a supplemental medical device. The catheter includes: a proximal end hole; a distal end hole that is positioned opposite the proximal end hole; a circumferential balloon that is located proximally of the distal end hole; an operational lumen that extends through the catheter from the proximal end hole to the distal end hole; a first bend that curves in a first direction; and a second bend that curves in a second direction that is generally opposite to the first direction, wherein the second bend is positioned distally of the first bend and proximally of the circumferential balloon. The first bend and the second bend are configured to brace the catheter against an arch of the blood vessel to inhibit recoil of the catheter. The supplemental medical device is configured for insertion into the blood vessel through the operational lumen of the catheter.

In some embodiments, the supplemental medical device may be configured as a hypotube.

In some embodiments, the supplemental medical device may be configured as a catheter.

In some embodiments, the supplemental medical device may support a stent.

In some embodiments, the supplemental medical device may include at least one balloon element.

In some embodiments, the supplemental medical device may include a first balloon element and a second balloon element that is spaced axially from the first balloon element.

In some embodiments, the supplemental medical device may include a stent and at least one balloon element.

In some embodiments, the at least one balloon element may include a first balloon element that is located distally of the stent and a second balloon element that is located distally of the first balloon element.

In some embodiments, the supplemental medical device may include a plurality of irrigation ports to facilitate fluid communication through the supplemental medical device into the blood vessel.

In some embodiments, the plurality of irrigation ports may include a first plurality of irrigation ports that are located proximally of the first balloon element and a second plurality of irrigation ports that are located distally of the second balloon element.

In another aspect of the present disclosure, a medical device is disclosed for treating a vascular narrowing within a blood vessel. The medical device includes: a catheter; a first supplemental medical device that is configured for insertion into the blood vessel through the catheter; and a second supplemental medical device that is configured for insertion into the blood vessel through the first supplemental medical device. The catheter includes a tubular body having a first bend curving in a first direction and a second bend curving in a second direction generally opposite to the first direction. The first bend and the second bend are configured to brace the catheter against an arch of the blood vessel to inhibit recoil of the catheter.

In some embodiments, the medical device may further include a guide wire.

In some embodiments, the catheter, the first supplemental medical device, and the second supplemental medical device may each be configured for insertion into the blood vessel over the guide wire.

In some embodiments, the first supplemental medical device may include a stent.

In some embodiments, the second supplemental medical device may include at least one balloon element.

In some embodiments, the at least one balloon element may include a first balloon element and a second balloon element that is located distally of the first balloon element.

In some embodiments, the second supplemental medical device may be configured such that the first balloon element and the second balloon element are positionable distally of the stent.

In some embodiments, the second supplemental medical device may include a plurality of irrigation ports to facilitate fluid communication through the second supplemental medical device into the blood vessel.

In some embodiments, the plurality of irrigation ports may include a first plurality of irrigation ports that are located proximally of the first balloon element and a second plurality of irrigation ports that are located distally of the second balloon element.

In another aspect of the disclosure, a medical device is disclosed for treating a vascular narrowing within a blood vessel. The medical device includes: a catheter; a first supplemental medical device that is configured for insertion into the blood vessel through the catheter; and a second supplemental medical device that is configured for insertion into the blood vessel through the first supplemental medical device, wherein the first supplemental medical device includes a stent and the second supplemental medical device includes at least one balloon element. The catheter includes a tubular body and a circumferential balloon that is secured to the tubular body. The tubular body includes a plurality of bends curving in a plurality of different directions such that the catheter is configured for bracing against an inner wall of the blood vessel to inhibit recoil of the catheter.

In some embodiments, the second supplemental medical device may include a first plurality of irrigation ports and a second plurality of irrigation ports that are located distally of the first plurality of irrigation ports.

In some embodiments, the first plurality of irrigation ports and the second plurality of irrigation ports may be configured to facilitate fluid communication through the second supplemental medical device into the blood vessel.

In some embodiments, the at least one balloon element may be positioned between the first plurality of irrigation ports and the second plurality of irrigation ports.

### BRIEF DESCRIPTION OF THE DRAWINGS

FIG. 1A is a cross-sectional view of one embodiment of a tube (tubular body) 1 of the described invention disposed in a blood vessel V (e.g., a bovine, Type III 7000 aortic arch), having the second segment resting on the arch 2000 fulcrum with a third bend 30 and fourth segment 400 deployed in left common carotid artery 5000 with a bovine origin. Also identified are descending aortic artery 1000, right subclavian artery 3000, right vertebral artery 3500, right carotid artery 4000, innominate (brachiocephalic) artery 6000, Type III arch 7000, left subclavian artery 8000, and left vertebral artery 8500.

FIG. 1B provides a cross-sectional view through tube 1 taken along line 1B in FIG. 1A according to an alternate embodiment of the disclosure.

FIG. 2 is a cross-sectional view of one embodiment of tube 1 of the described invention in place having the second segment 200 resting on the fulcrum of arch 2000 with an obtuse (inner) third bend 30 and fourth segment 400 deployed in right subclavian artery 3000.

FIG. 3A is a cross-sectional view of one embodiment of tube 1 of the described invention in place having the second segment 200 resting on the fulcrum of arch 2000 with a second bend 20, and third segment 300 deployed in the right carotid artery 4000.

FIGS. 3B and 3C are enlargements of the areas of detail identified in FIG. 3A.

FIG. 4 is a cross-sectional view of an exemplary embodiment of tube 1 having a final element 400 comprising multiple bends and segments deployed in the right subclavian artery 3000.

FIG. 5 is a cross-sectional view of one embodiment of the described invention in place, having tube 1 having at least one lumen, which is the interior of tube 1, said tube 1 having a first segment 100 entering from an arm vessel or axillary artery or vein; second segment 200 disposed between first bend 10 and second bend 20, resting on the fulcrum of arch 2000 and third segment 300 deployed in the left common carotid artery 5000.

FIG. 6 is a cross-sectional view of one embodiment of tube 1 of the described invention in place having tube 1 having at least one lumen, which is the interior of tube 1, said tube 1 having a first segment 100 entering from an arm vessel or axillary artery or vein through left subclavian artery 8000, optional first side hole 170 proximal to the entry point of left vertebral artery 8500, second segment 200 resting on aortic arch 2000, second segment including optional side hole 270, second bend 20 directing third segment 300 upward into innominate (brachiocephalic) artery 6000 wherein end hole 405 is disposed. In this embodiment, second segment 200 includes optional side hole 270.

FIG. 7 is a cross-sectional view of the embodiment of FIG. 5 of the described invention, in place further including at least two additional side holes 170 and 171 disposed within right subclavian artery 3000; further including optional at least one balloon element 333 proximal to end hole 405 disposed within left subclavian artery 8000, and an inflation lumen (not shown) for balloon element 333, said inflation lumen passes from the exterior of a body and extends substantially through the wall of the intraluminal portion of tube 1 to balloon element 333.

FIG. 8 illustrates the angle ranges for bend 10 (i.e., angle range 190 degrees to 280 degrees) and the angle ranges for bend 20 (i.e. angle range 70 degrees to 150 degrees), wherein the opposite angle range for bend 10 is 1111 and the opposite angle range for bend 20 is 2111. The arrow in FIG. 8 denotes direction of passage of devices from outside the body direction relative to the angles 1111 and 2111. It should be noted that bend numbers 10 and 20 have corresponding, opposing angle ranges such as 1111 and 2111, respectively. This nomenclature distinction has been to insure clarity of disclosure.

FIG. 9 illustrates an arm access arch fulcrum support catheter with three bends. In addition to bend 10 and bend 20 a third bend 30 is disclosed. Beyond bend 30 is segment 400.

FIG. 10 shows an illustration of the preferred embodiment for transfemoral percutaneous treatment of the right carotid in most anatomies, with the balloon 333 deflated. Said balloon 333 is disposed at (or adjacent to) the distal end hole 405. More particularly, first internal curve 3111 of first bend 10 has a curvature of 70 to 120 degrees; second internal curve 4111 of second bend 20 has a curvature of 65 to 130 degrees. To reiterate, it should be noted that bend numbers 10 and 20 have corresponding, opposing angle ranges or internal curves such as 3111 and 4111, respectively. This nomenclature distinction has been to insure clarity of disclosure.

FIG. 11 illustrates a preferred embodiment for the left carotid artery with the balloon 333 inflated. In this embodiment, internal curve 5111 of bend 10 has a curvature of between 60 and 120 degrees.

FIG. 12 illustrates external elements of the present invention, including filter 9222, Y-connector 9223, external termination device of first catheter such as a Luer Lock element 9224, venous sheath 9225, flow regulator 9226, and stopcock 9227.

FIG. 13 illustrates an irrigation catheter 9300, having a distal end 9333, and a plurality of fluid-delivery ports 9334 (depicted in position in FIG. 18).

FIG. 14 depicts an elongated embodiment of the irrigation catheter 9300 of FIG. 13, further including angioplasty balloon element 9555, occlusion balloon element 9556, including the plurality of optional irrigation ports 9334, further having said optional irrigation ports 9334 disposed proximal to optionally tapered distal tip 9557.

FIG. 15 illustrates the interior of a narrowed internal carotid-artery lumen 7892, showing a balloon 333 disposed proximal to distal end hole 405 of tube 1 and narrowed area 7892, with blood-flow direction depicted by arrows. In particular, the narrowed lumen 7892 is restricted by cholesterolic plaque 7891 extending from carotid bulb 7890.

FIG. 16 illustrates the narrowed internal carotid-artery lumen 7892 of FIG. 15, additionally disclosing a delivery catheter 8970 that supports and is capable of delivering a stent 8971 over a delivery wire 8972, further showing flow direction at the lesion by the arrow.

FIG. 17 illustrates the narrowed internal carotid-artery lumen 7892 of FIG. 15, additionally disclosing an angioplasty occlusion balloon 8973 (e.g., a first balloon element), disposed upon fluid delivery catheter (hypotube) 8974, blood flow direction shown by arrows.

FIG. 18 illustrates the narrowed internal carotid-artery lumen 7892 of FIG. 15, further depicting delivery wire 8972, which is shown guiding irrigation catheter 9300 having optional, multiple fluid-delivery ports 9334 (one shown in FIG. 13) disposed thereon both proximally to temporary occlusion (second) balloon element 8975, and distally to distally delivered deflated angioplasty (first) balloon element 8973, wherein balloon elements 8973 and 8975 are mounted on irrigation catheter 9300. More specifically, (first) balloon element 8973 is located distally of, and is spaced axially from, (second) balloon element 8975 along the length of the irrigation catheter 9300. Stent 8971 is illustrated as being exposed (delivered, unsheathed) from hypotube 8970i, which may replace or supplement delivery catheter 8970. Hypotube 8970i and irrigation catheter 9300 each have a sufficient diameter to be able to be advanced over delivery wire 8972.

### DETAILED DESCRIPTION OF THE INVENTION

Referring now to the drawings wherein like reference numerals identify similar structures, element, and features, various embodiments of the presently disclosed systems and devices will be discussed.

The term “recoil and displacement”, as used herein refers to the phenomenon of catheter prolapse or displacement (slipping forward, back, or down, and out of the desired position) due to a counterforce against the catheter by the advancing wire, second catheter, or other, additional device.

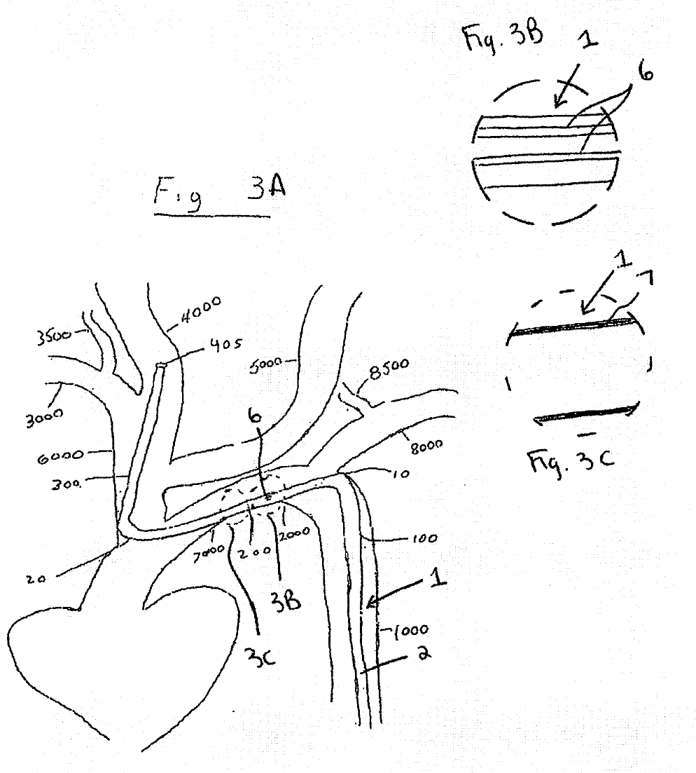

The current invention's novelty, in some embodiments, rests upon the use of an anatomical fulcrum as an anti-kickback, anti-displacement support structure. Beyond the shaping of the invention to allow said support, the invention in some embodiments deploys a final element at the distal end to facilitate delivery of said distal end to the target area. The final element of the simplest embodiment of the invention is shown in FIG. 3A.

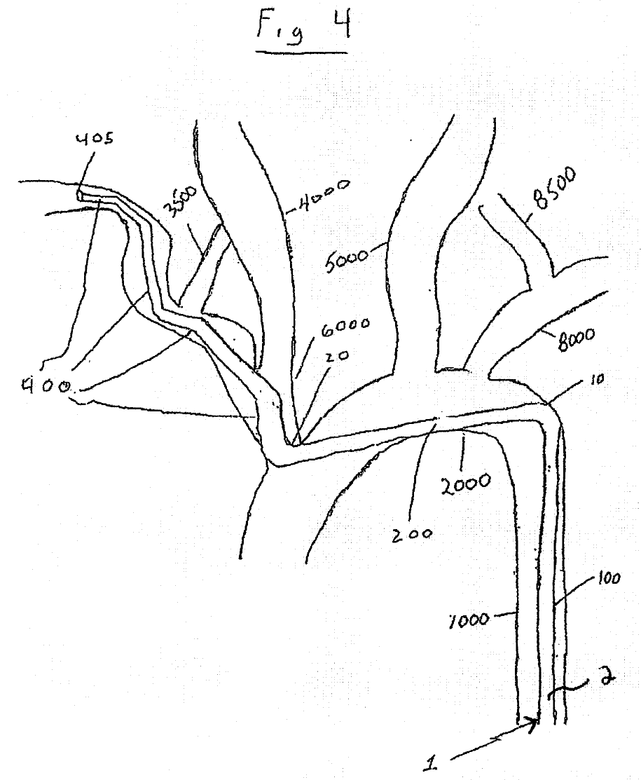

The final element in the preferred embodiment comprises two bends, three segments and one end hole. The final element may comprise one or more additional bends and one or more additional segments beyond those comprising the preferred embodiment. The final-element configuration is determined by the path the user of the invention determines is necessary to deliver the distal end hole 405 of the current invention to the target area as illustrated in FIG. 4. Additionally, although most commonly the second segment will rest on a vascular fulcrum, any segment, or in some cases multiple segments, may utilize a vascular anatomical structure for securement, to prevent unwanted kickback and displacement. Additionally, the preferred embodiment for the current invention will have an additional circumferential balloon near its distal end hole, with at least one additional lumen to inflate and deflate said balloon positioned substantially within the wall of said catheter, in its intraluminal segment. Not pictured is a proximal branching of said additional lumen to its own proximal end hole at its own external termination device, said branching which occurs outside the patient's body, which is well known in the prior art. Inflation of said balloon is capable of completely occluding the ipsilateral common carotid artery proximal to the target stenosis, which is typically in the internal carotid artery, thereby allowing reversal of flow in said internal carotid artery when flow is allowed through the (main) catheter of the current invention. This flow can be active or passive. It can be aided in some cases by proximal aspiration, vacuum, or other pump mechanism. It can be aided in some cases by differential pressure between the target artery and a vein to which a circuit of flow is subsequently established. Said vein can be accessed with a secondary (separate) catheter, and tubing can connect the proximal end of said (main) catheter with the proximal end of said secondary (separate) catheter. In some embodiments a regulator along said tubing can modulate flow rates as well. In some embodiments a filter along said tubing can filter the blood and remove embolic debris before returning it to the patient as well. In some embodiments flow reversal can also be augmented by infusion of fluids across and/or distal to said target lesion, as previously described with similar devices by Walzman. Such infusions can also act to decrease a known sump effect from the brain while flow is reversed across the lesion, thereby decreasing the potential for ischemia of the brain tissue that can result from said sump effect.

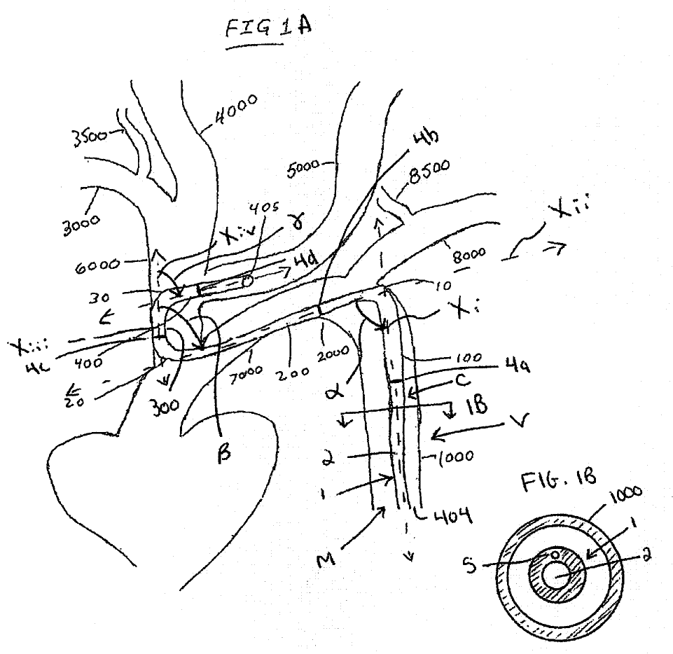

Now referring to FIG. 1A, a medical device M (e.g., a catheter C) is disclosed that includes the tube 1 of the present invention. The tube 1 includes a proximal end hole 404 and the aforementioned distal end hole 405, which is positioned opposite to the proximal end hole 404. The catheter C is shown deployed in the aorta with distal end hole 405 terminating in an abnormal anatomical variation of the left carotid artery 500 referred to as a bovine arch. The device of this embodiment the current invention has seven principal elements. The first three of said elements are bends, and four are segments of the tube. More particularly, first bend 10 connects segment one 100 to segment two 200 at a non-obtuse angle α, as measured as an angle from the proximal catheter tubing to the tubing of the second segment, in this example (e.g., such that the angle α (FIG. 1A) is defined between respective longitudinal axes Xi, Xii of segments 100, 200). It is envisioned, however, that segments 100, 200 may be configured and positioned so as to achieve any necessary or desired angle α (e.g., depending upon the particular nature of the procedure being performed, spatial restrictions dictated by the patient's vasculature, etc.).

First bend 10 extends in a first direction and may be active or passive. A passive bend, as disclosed by the prior art, is a bend which is pre-formed by the use of a wire or a braid. A passive bend 10 has been treated in such a way prior to the introduction to the body that, if there are no other forces action, it will form a desired (e.g., non-obtuse) angle. In order to deploy a tube must be straight, so there must be a force to straighten bend 10, such as a wire, a stiff inner or outer tube or combination, such that upon removal of said external force, the desired (e.g., non-obtuse) angle is formed. In other embodiments, any bend may be active or passive. In some embodiments, all bends are active; in other embodiments all bends are passive; in yet other embodiments, bends may be a mix of active bends and passive bends.

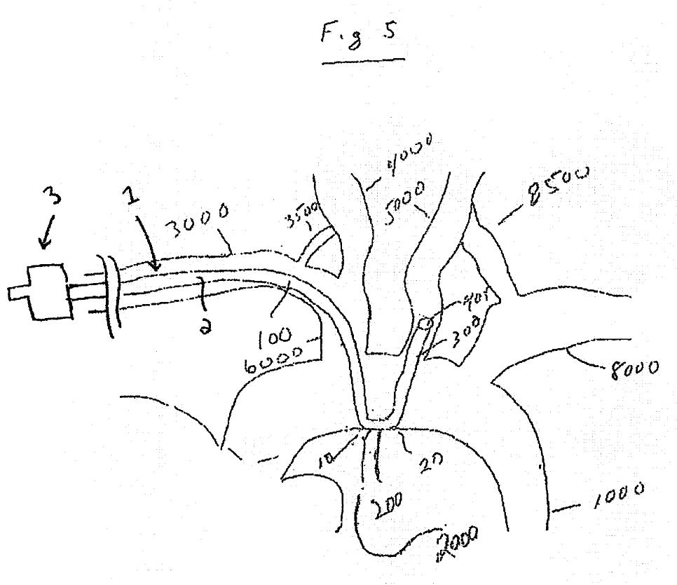

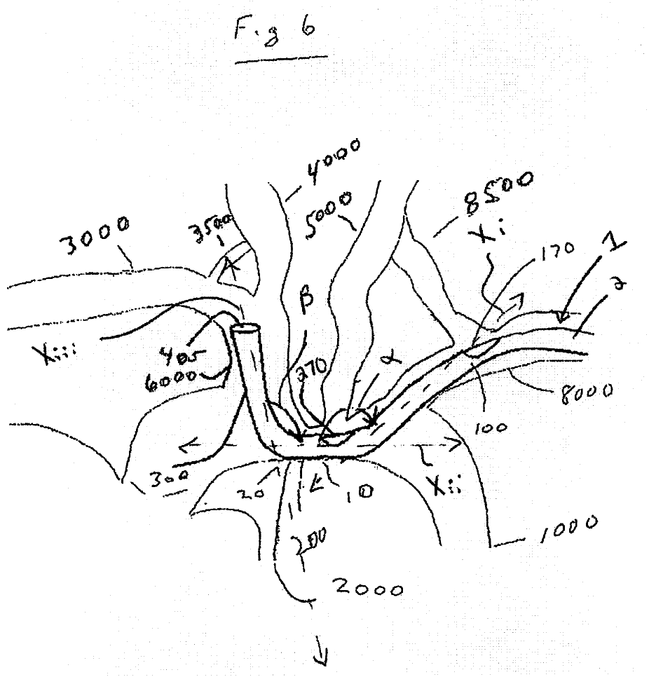

Other embodiments are adapted to access aortic arch 2000 through a vessel in the arm or, for example, from a radial artery, brachial artery, axillary artery (or vein) (not shown), when such access may be preferred. In an embodiment depicted in FIG. 5, tube 1 includes at least one operational (primary, working) lumen 2 that extends through the catheter C from the proximal end hole 404 to the distal end hole 405. In FIG. 5, first segment 100 is shown accessing aortic arch 2000 through right subclavian artery 3000. FIG. 5 further illustrates tube 1 having second segment 200 disposed between first bend 10 and second bend 20, resting on the fulcrum of arch 2000 and third segment 300 deployed in the common carotid artery 5000. As seen in FIG. 6, it is envisioned that the second segment 200 may include an optional side hole 270 that is located between bends 10, 20. For example, it is envisioned that side hole 270 may be configured to allow for the passage of a supplemental medical device (e.g., a catheter, a deburring device, etc.) therethrough.

In a variant embodiment of FIG. 5, segment 100 of tube 1 is shown as having a “gentle curve” nearest to an external termination device 3 (e.g., a Luer lock) and at least two bends 10 and 20.

FIG. 6 illustrates tube 1 of the described invention in place in the body, wherein second segment 200 rests on aortic fulcrum 2000. Aortic fulcrum 2000 is accessed via an arm vessel or axillary artery or vein from the opposite arm illustrated in FIG. 5. In the embodiment of FIG. 6, tube 1 passes through left subclavian artery 8000. In the illustrated embodiment optional at least one side hole 170 is disposed proximal to left vertebral artery 8500. As seen in FIG. 6, the side hole 170 is formed in segment 100 and is located proximally of bend 10. To properly access the vasculature, it is envisioned that the segment 100 may be either (generally) linear, as seen in FIG. 4, for example, or that segment 100 may include a non-linear configuration defining a curvature, as seen in FIG. 6, for example. Second segment 200 rests on aortic fulcrum 2000 and extends from segment 100 such that the angle α is obtuse. Second segment 200 further includes side hole 270 disposed proximal to aortic fulcrum 2000 and between bends 10, 20. Second bend 20 directs third segment 300 upward into innominate artery 6000 wherein end hole 405 is disposed. Third segment 300 extends from second segment 200 such that an β (FIG. 6) is defined between respective longitudinal axes Xii, Xiii of segments 200, 300. In the illustrated embodiment, segments 200, 300 are configured and positioned such that angle β is obtuse and such that second bend 20 extends in a second direction that is (generally) opposite to the first direction of first bend 10. It is envisioned, however, that segments 200, 300 may be configured and positioned so as to achieve any necessary or desired angle β (e.g., depending upon the particular nature of the procedure being performed, spatial restrictions dictated by the patient's vasculature, etc.).

The combination of a catheter that utilizes the inferior curve of the aortic arch as a vascular fulcrum with optional side holes, through which additional catheters can be passed, may further facilitate catheterization of bilateral vertebral and carotid arteries via a single access sight in either arm.

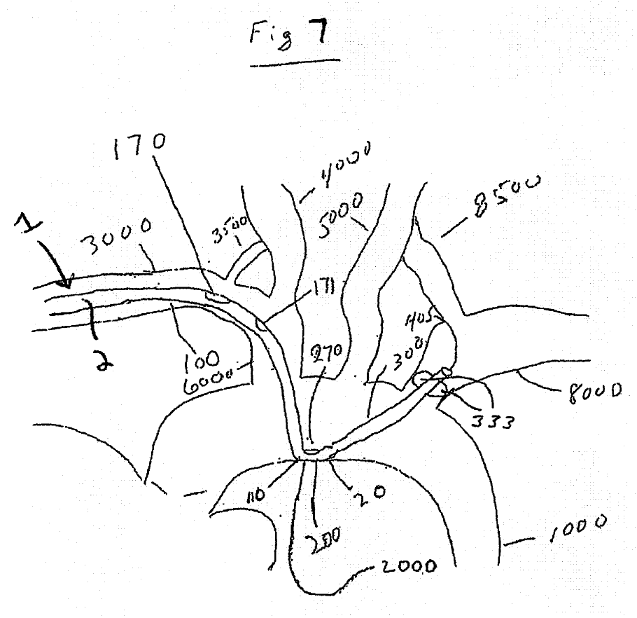

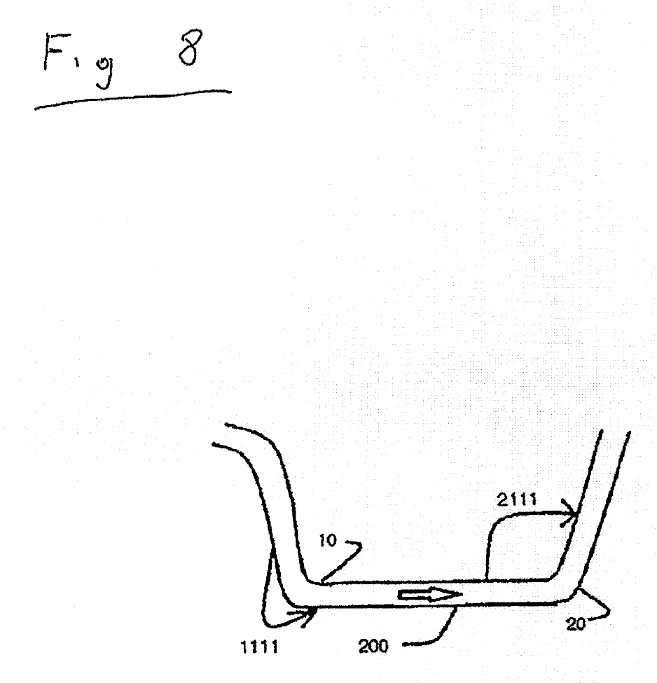

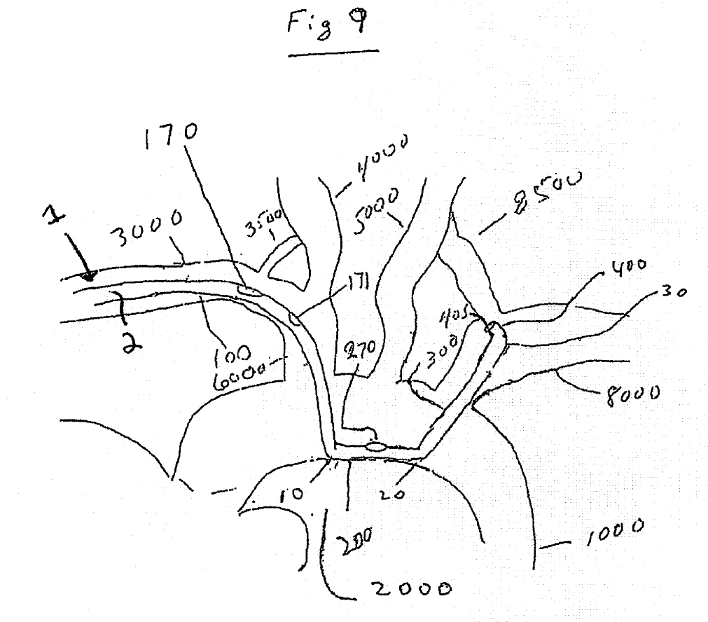

As depicted in FIG. 7, another embodiment also accesses aortic arch 2000 through a vessel an arm. In the embodiment of FIG. 7, tube 1 includes lumen 2, a first segment 100 that is configured to access aortic arch 2000 through right subclavian artery 3000. FIG. 7 further illustrates tube 1 having the side hole 270 disposed upon second segment 200 within aortic arch 2000 and a side hole 170 disposed upon first segment 100 deployed within right subclavian artery 3000 proximal to the entry point of right vertebral artery 3500, and an additional third optional side hole 171 proximal to the origin of the right common carotid artery. As discussed in connection with side holes 170, 270, it is envisioned that side hole 171 may be configured to allow for the passage of a supplemental medical device (e.g., a catheter, a deburring device, etc.) therethrough.

It should be noted that in all embodiments containing more than one balloon, each balloon may optionally require a separate inflation lumen.

Other variants of catheter embodiments optionally include at least one valve 4. For example, as seen in FIG. 1A, tube 1 may include a first valve 4a located within segment 100 proximally of bend 10; a second valve 4b located within segment 200 distally of bend 10 and proximally of bend 20; a third valve 4c located within segment 300 distally of bend 20 and proximally of bend 30; and/or a fourth valve 4d located within segment 400 distally of bend 30 and proximally of end hole 405. It is envisioned that each of the valves 4 may be configured to receive a medical device in sealed engagement. It is also envisioned that each of valves 4 may be biased towards a closed position to regulate blood flow through one or more segments 100, 200, 300, 400 of tube 1. Still other variants of catheter embodiments may optionally include at least one supplemental irrigation lumen 5 (FIG. 1B) substantially in the wall of tube 1, which may include an end hole terminating either inside or outside tube 1. It is envisioned that lumen 5 may be configured to communicate an irrigation fluid through tube 1 to help minimize clot formation in the exit region adjacent to a target area in the vasculature.

In an alternative embodiment, materials or techniques may be employed so as to achieve any desired configuration for bends 10, 20, 30. For example, materials may be chosen and techniques utilized such that bends 10, 20, 30 are obtuse, non-obtuse, or at approximately right angles. Such embodiment may include the incorporation of shape-memory metals or polymers. In addition or in the alternative, radiation may be focused on a point of tube 1 such that bend 10 is forced to adopt a desired, non-obtuse angle of segment two relative to the proximal catheter of segment one to position segment two 200 over the fulcrum of aortic arch 2000.

Segment one 100 has a length of at least (approximately) 20 cm in length and an internal diameter of from (approximately) 0.1 French to (approximately) 30 French. In a preferred embodiment deployed transfemorally for access of the innominate arteries distal branches with a Type II and Type III arch, first bend 10 is deployed in the artery such that angle α is non-obtuse so as to orient segment two 200 for optimal positioning on the fulcrum of aortic arch 200.

Segment two 200 measures at least (approximately) 3 cm in length and no more than (approximately) 35 cm in length in the preferred embodiment of FIG. 3A. Segment two 200 has an internal diameter of from (approximately) 0.1 French to (approximately) 30 French. Segment two 200 has a first end which terminated in first bend 10 and a second end which terminates in second bend 20. It is envisioned that segments 200, 300 may be configured and positioned such that angle β lies substantially within the range of (approximately) 30 degrees to (approximately) 150 degrees.

Second bend 20 connects to segment three 300 of tube 1. Segment three 300 measures at least (approximately) 0.5 cm in length and has an internal diameter of from(approximately) 0.1 French to (approximately) 30 French. Segment three 300 has a first end which terminates in second bend 20 and connected to segment two 200 of tube 1, and a second end terminating at third bend 30.

Third bend 30 connects to segment four 400 (FIG. 4) of tube 1 and extends in a third direction different from the first direction (of first bend 10) and the second direction (of second bend 20). Segment 400 extends from second segment 300 such that an angle γ (FIG. 1A) is defined between respective longitudinal axes Xiii, Xiv of segments 300, 400. In the illustrated embodiment, segments 200, 300 are configured and positioned such that angle γ is approximately 90 degrees. It is envisioned, however, that segments 300, 400 may be configured and positioned so as to achieve any necessary or desired angle γ (e.g., depending upon the particular nature of the procedure being performed, spatial restrictions dictated by the patient's vasculature, etc.). For example, it is also envisioned that segments 300, 400 may be configured and positioned such that angle γ is acute or obtuse.

Segment four 400 measures at least (approximately) 0.5 cm in length and has an internal diameter of from (approximately) 0.1 French to (approximately) 30 French. Segment four 400 has a first end which terminates in third bend 30 and connected to segment three 300 of tube 1, and a second end terminating at distal hole 405.

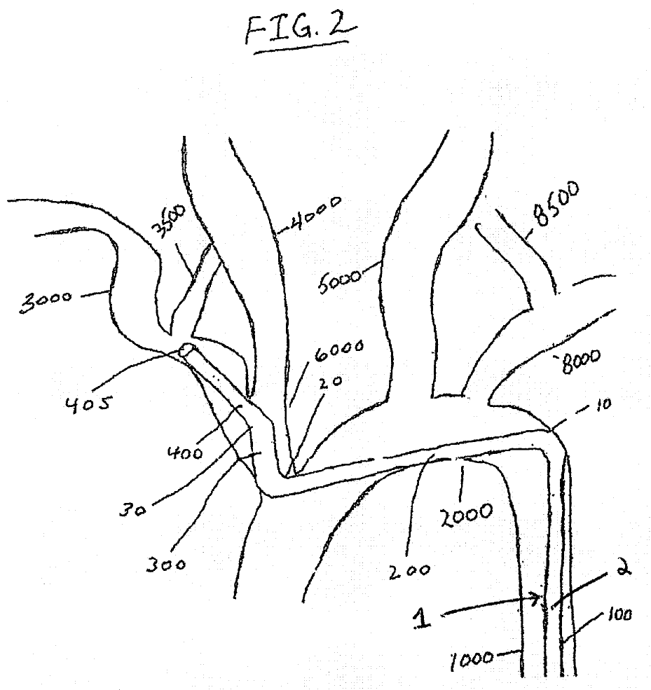

Now referring to FIG. 2, the present invention is shown deployed in a Type III aortic arch anatomy. Segment one 100 is deployed downwardly in the ascending aorta 1000, which is located below the fulcrum formed by the arch of the aorta 2000. The middle of segment two 200 is shown resting on the fulcrum formed by the arch of the aorta 2000. Segment three 300 is shown in this example being upwardly deployed into innominate artery 6000, and segment four 400 extending upwardly from third bend 30 at an obtuse angle, relative to the catheter of segment three 300, and deployed distally in right subclavian artery 3000.

Now referring to FIG. 3A, the present invention is shown deployed in a sample aortic arch anatomy. Segment one 100 is deployed downwardly in the ascending aorta 1000, which is located below the fulcrum formed by the arch of the aorta 2000. The middle of segment two 200 is shown resting on the fulcrum formed by the arch of the aorta 2000. In this embodiment the first bend 10 (e.g., angle α) is shown as being non-obtuse. While this is a feature of this embodiment, a non-obtuse bend is not a limitation of the present invention, rather a shape limitation of a particular embodiment which will allow the use of the arch of the aorta 2000 to prevent kick-back and displacement in particular anatomical scenarios. An obtuse bend will not allow the use of the arch of the aorta 2000 to prevent kick-back and displacement while obtaining transfemoral access to the right carotid artery in these select anatomical variants.

According to one embodiment, the middle segment 200 includes ridges 6 (FIGS. 3A, 3B). to promote stability at the focal point 2000. Although shown as extending longitudinally (e.g., in (generally) parallel relation to axis Xii (FIG. 1A), it is also envisioned that ridges 6 may extend transversely (e.g., in (generally) orthogonal relation to axis Xii (FIG. 1A)). Additionally, while segment 200 is illustrated as including two ridges 6 in the illustrated embodiment, it should be appreciated that the number of ridges 6 may be increased or decreased in alternate embodiments without departing from the scope of the present disclosure. For example, embodiments in which segment 200 includes a single ridge 6 or three (or more) ridges 6 are also contemplated herein.

According to another embodiment, the middle segment two 200 is coated with an elastic material 7 (FIG. 3C) to deform adjacent to (e.g., atop) the fulcrum point 2000 for improved securement.

The various components of the described invention may be comprised of one or more materials. Thermoplastics include, but are not limited to, nylon, polyethylene terephthalate (PET), urethane, polyethylene, polyvinyl chloride (PVC) and polyether ether ketone (PEEK).

Thermosets include, but are not limited to, silicone, polytetrafluoroethylene (PTFB) and polyimide. Composites include, but are not limited to, liquid crystal polymers (LCP). LCPs are partially crystalline aromatic polyesters based on p-hydroxybenzoic acid and related monomers. LCPs are highly ordered structures when in the liquid phase, but the degree of order is less than that of a regular solid crystal. LCPs can be substituted for such materials as ceramics, metals, composites and other plastics due to their strength at extreme temperatures and resistance to chemicals, weathering, radiation and heat. Non-limiting examples of LCPs include wholly or partially aromatic polyesters or co-polyesters such as XYDAR® (Amoco) or VECTRA® (Hoechst Celanese).

According to some embodiments, the bends comprise a shape memory polymer (SMP). Shape memory polymers include, but are not limited to meth-acrylates, polyurethanes, blends of polystyrene and polyurethane, and PVC. According to some embodiments, the bends of the catheter comprises a shape memory alloy (SMA). Non-limiting examples of shape memory alloys include nickel-titanium (i.e., nitinol).

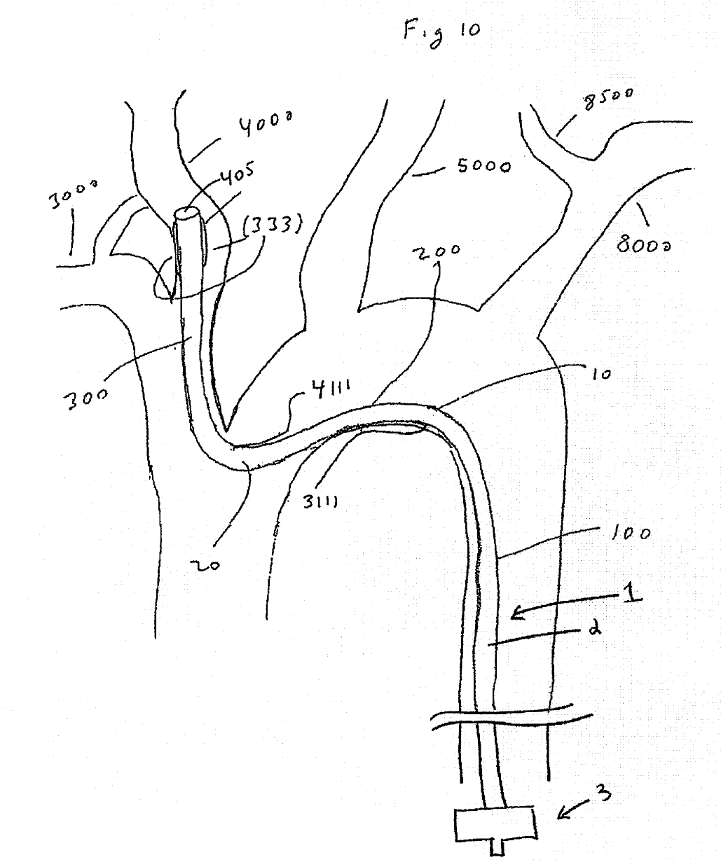

Now referring to FIG. 10 which discloses a preferred embodiment for the right carotid the first segment 100 extends from the external termination device 3 (e.g., the aforementioned Luer lock) through first curve 3111 of bend 10. In the current embodiment, the distal region of the first segment 100, at first curve 3111, and extending into second curve 4111 of bend 20, which extends into the second segment 200 and curves in a substantially opposite direction to first curve 3111, are optimized to rest upon the lesser curve of the aortic arch, thereby providing support, and bracing the catheter C within the blood vessel V so as to inhibit (if not entirely prevent) recoil (e.g., kickback, prolapse, etc.) of tube 1 and any additional (supplemental) medical devices that are subsequently passed through tube 1 into the distal vasculature, examples of which are discussed below. It should be noted that second segment 200 is bounded proximally by first bend 10 and first curve 3111, and distally by second bend 20 and second curve 4111.

In certain embodiments, first segment 100 of tube 1 may having an effective length (segment within the body) that lies substantially within the range of (approximately) 30 cm to (approximately) 70 cm, and second segment 200 of tube 1 may have an effective length that lies substantially within the range of (approximately) 4 cm to (approximately) 25 cm when used transfemorally for carotid bifurcation pathology. The tube 1 may include an outer diameter that lies substantially within the range of (approximately) 4 Fr to (approximately) 12 Fr for this application. The tube 1 additionally has at least one circumferential balloon 333 near (e.g., at or (generally) adjacent to) its distal end hole 405, which is optimized for atraumatic temporary occlusion of the common carotid artery during angioplasty and stenting, in order to create flow reversal across the lesion. The (primary working) lumen 2 of tube 1 includes an internal diameter sufficient to allow for the insertion and delivery of additional (e.g., supplemental) medical devices (such as balloons, hypotubes, wires, stents, etc.). The tube 1 may also include at least one additional lumen (e.g., the aforementioned irrigation lumen 5 (FIG. 1B), which extends within the wall of the tube 1 in (generally) parallel relation to the lumen 2 along all or a portion of the effective length of said tube 1 to facilitate inflation and deflation of said at least one circumferential balloon 333. It is envisioned that, in certain embodiments, bend 10 (e.g., first curve 3111) may lie substantially within the range of approximately 60 degrees to approximately 120 degrees, and that, in certain embodiments, the bend 20 (e.g., second curve 4111) may lie substantially within the range of approximately 65 degrees to approximately 130 degrees. As seen in FIG. 10, for example, bends 10, 20 curve in (generally) opposite directions. To facilitate insertion of tube 1 into the vasculature, it envisioned that a straight inner dilator may be used to substantially straighten said tube 1, as is well known in the prior art/field.

Like the prior art described above, the current invention relies on flow reversal across the lesion during angioplasty and stenting to minimize the risks of thromboembolic ischemic complications during the procedure. However, whereas the cited prior art relies on a carotid open surgical cut-down, the current invention optimally uses percutaneous techniques. The current invention additionally, in the preferred transfemoral embodiment, utilizes vascular fulcrums for support of the devices, to reduce potential complications and risks. Furthermore, as previously described by Walzman, the current device additionally optionally utilizes infusion of fluid distal to the lesion during the procedure to aid in flow reversal across the lesion, while minimizing a sump effect from the brain that can contribute to ischemic complications. In order to accomplish this, these embodiments of the current invention optimally utilize a hypotube capable of irrigation, in addition to its role as an access rail for balloons mounted on their delivery catheters as well as stents mounted on their respective delivery catheters, and/or additional balloon catheters capable of irrigation as well. In other embodiments the current invention may deploy an additional temporary balloon to occlude the vessel distally.

The current invention also optionally utilizes angioplasty balloons on novel catheters that can also irrigate, and or additional irrigation catheters. Additionally, the current invention optionally utilizes a double balloon catheter, wherein one balloon is optimized for angioplasty and at least one additional balloon is optimized for atraumatic temporary balloon occlusion of a vessel. In this way, an angioplasty balloon can be advanced over a wire, said wire optionally having an inner lumen and distal end or/and side holes for irrigation, said angioplasty can be inflated across said lesion to dilate the stenosis, and then deflated. The occlusion balloon can be proximal or distal to the angioplasty balloon; in the preferred embodiment it is proximal. To reduce the number of exchanges necessary during the procedure, each of which can increase risks, said balloon can then optionally be advanced past said lesion and not removed. Said second balloon temporary occlusion balloon can then be inflated distal to said lesion, further decreasing the potential for a sump of blood flow from the brain during the procedure.

Additional fluids can then be infused through said double balloon catheter, with egress ports optionally both proximal and optionally distal to said occlusion balloon, to aid in flow reversal across the lesion proximally, and prevent clot formation distal to the occlusion balloon during balloon occlusion. Said balloon, a conventional single angioplasty balloon, and/or said irrigation catheter 9300 (or hollow wire capable of irrigation) can further optionally have a detachable hub, which is an additional novelty of the current invention. Said optional detachable hub can have pressure-mounted design or a threaded-screw design, or others.

Threaded screw designs can include a thread on the inside of the detachable hub and a corresponding opposite thread on the outside of the proximal end of the catheter, or alternatively the thread can be on the outside of the distal side of the hub and on the inside of the proximal end of the catheter. This removable hub (not shown) will allow these devices to be used as a rail (like a wire) to deliver additional catheters, such as an angioplasty balloon mounted catheter or a stent delivery catheter, both in an “over-the-wire” configuration and in a “rapid exchange” configuration, by allowing said additional catheters to be loaded over the proximal end of these catheters after the hub is detached. Said catheters can additionally have optional valves in order to prevent deflation of a temporary occlusion balloon during hub detachment. The hub can be re-attached to allow continuation of fluid delivery and/or balloon deflation when desired.

All described catheters and wires can have tapered or non-tapered distal ends.

Stents can be self-expanding, balloon expanded, or a hybrid.

The current invention may also optionally include a plug or balloon to occlude the external carotid artery, to further ensure flow is reversed across the stenosis in the internal carotid artery during angioplasty and stenting. Said plug or balloon may be mounted on a wire or catheter, may be detachable or non-detachable, may be retrievable or non-retrievable, may be permanent or temporary. One example of a temporary detachable plug is a biodegradable hydrogel plug, which the body can recanalize.

In an optional embodiment, the device of the present invention further comprises at least one vascular plug, capable of obstructing collateral flow from a branch such as the external carotid artery. Said plug is preferably located between at least one circumferential balloon and a vascular blockage, to further ensure flow is reversed at the obstruction during angioplasty and stenting. It should be noted that in one embodiment, a patient's body will break down the plug and restore flow in a vascular branch over a set period of time.

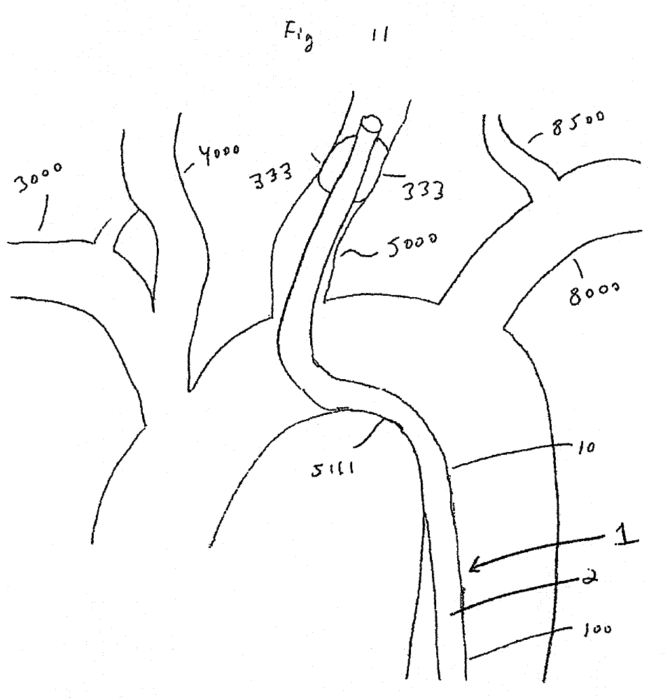

Now referring to FIG. 11 which discloses the preferred embodiment for the left carotid. The left carotid and right carotid arteries are sized in accordance with the anatomical dimensions of said arteries. Said dimensions vary from patient to patient but are readily determinable. Accordingly, embodiments intended for left carotid use will be sized as described above for the right carotid artery, with adjustments for these variations.

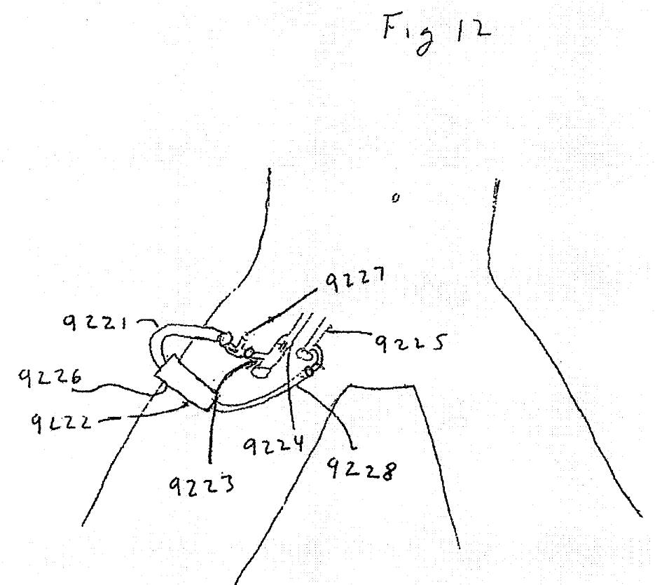

Now referring to FIG. 12 which illustrates the external elements of the present invention, more particularly illustrating that approach can be right femoral artery and/or left femoral artery (right illustrated). Radial, brachial, or axillary arterial access, and other percutaneous ports of access, can be used as well. For example, to inhibit blood loss, said external elements include some or all of filter 9222, Y-connector 9223, external termination device of arch-fulcrum catheter such as a Luer Lock element 9224, venous sheath 9225, flow regulator 9226, and stopcock 9227; and tube elements 9221 and 9228. During use of the medical device M, blood flow may be reversed in the target blood vessel V (e.g., artery) such that blood flows through (and from) the medical device M. This blood can either be discarded (if the volume is negligible) or re-circulated to the patient. In those instances of recirculation, prior to returning to the patient (e.g., via the venous sheath 9225), it is envisioned that blood may be passed through the filter 9222 to remove debris. When necessary or desirable, directing blood flow through the filter 9222 and the venous sheath 9225 may create a passive flow mechanism in that blood flow may be directed from a (higher pressure) artery, through the medical device M, and into a (lower pressure) vein. Should higher velocities and/or volumes of flow reversal be necessary or desirable, such as, for example, when the lumen 2 extending through the tube 1 is partially obstructed (e.g., via stent 8971, irrigation catheter 9300, etc.), it is envisioned that the flow velocity and/or the flow rate may be augmented using a pump or a vacuum.

Alternatively, venous sheath 9225 can be used in any vein of sufficient size. It should also be noted that the flow regulator 9226 can be any one previously disclosed by the prior art: a wheel on a ramp (like a standard), or can involve routing blood through a higher or lower resistance path. Alternatively, the regulator can be active, utilizing pumps, artificial pressure gradients, vacuums, or other mechanisms that can increase flow through a narrow path when desired, thereby allowing a smaller sized delivery catheter to still effect flow reversal during device delivery, thereby reducing potential for access site complications, and increasing available ports of entry.

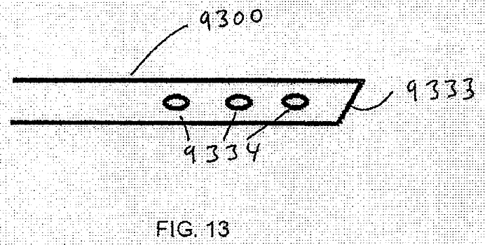

FIG. 13 illustrates one example of a (first) supplemental medical device that is configured for insertion into the blood vessel V through the catheter C. More specifically, the supplemental medical device is shown as the aforementioned irrigation catheter 9300, which includes a distal end 9333 and a plurality of irrigation ports 9334.

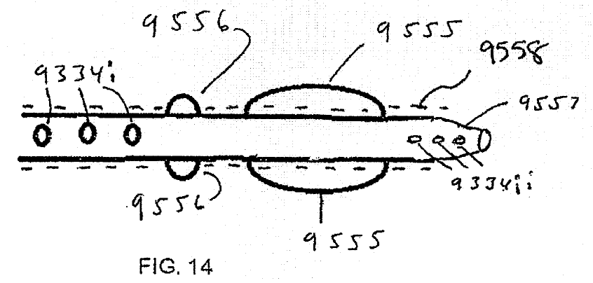

FIG. 14 depicts an elongated embodiment of the irrigation catheter 9300 of FIG. 13, further including angioplasty balloon element 9555, occlusion balloon element 9556, and irrigation ports 9334. In the illustrated embodiment, the irrigation catheter 9300 includes a first plurality of ports 9334i that are located proximally of the occlusion balloon element 9556 and a second plurality of ports 9334ii that are located distally of angioplasty balloon element 9555. As seen in FIG. 14, it is envisioned that the irrigation catheter 9300 may include a tapered distal tip 9557 and that the occlusion balloon element 9556 may include a transverse cross-sectional dimension (e.g., a diameter) less than that of the angioplasty balloon element 9555. A still further embodiment also optionally comprises a “peel away sheath” 9558 to protect the access artery from the balloons 9555 and/or 9556, and the balloons 9555 and/or 9556 from the access artery and tissue, during insertion of the tube 1 and the balloons 9555, 9556. The peel-away-sheath 9558 can be very thin, and the tube 1 can optionally have a slightly larger outer diameter to prevent leakage around it after the peel-away sheath 9558 is removed after the balloons 9555 and/or 9556 are positioned intravascularly.

In a still further embodiment, the disclosed medical device M further includes at least one of series of angioplasty balloons and/or stent delivery catheters with removeable hubs and/or side ports. Said series can be delivered over each other, such that a first delivery wire “rail” crosses a lesion. Then an angioplasty balloon is inflated, with flow reversed, optionally aided by active pumps or similar. As such, the current invention can have the hub and side port of the angioplasty balloon be removable. The method simply requires that the user advance the balloon, after angioplasty inflation and subsequent deflation, slightly past a target blockage. Then the user slides the next balloon catheter, or the stent catheter, over the balloon catheter. In the prior art, systems require exchanging the balloon catheter for another larger balloon or the stent. This maneuver enhances risk to patients; for example, the wire can move, the time for the procedure be increased, and/or an increased loss of blood.

In said embodiment, the angioplasty balloon catheter is sometimes further capable of delivering fluid, which can be delivered distal to the blockage and/or across the blockage. Thereby, the any potential “sump effect” of blood flow diversion from the distal tissue is reduced, while flow is reversed across the blockage.

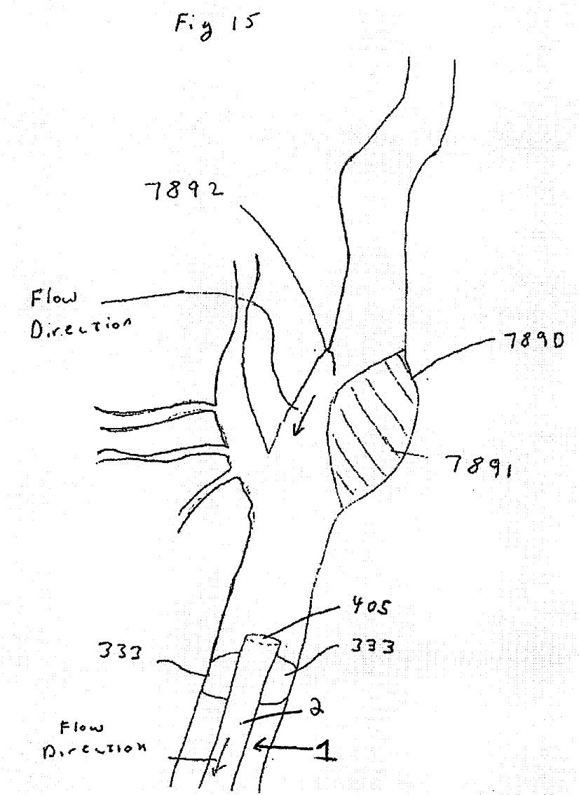

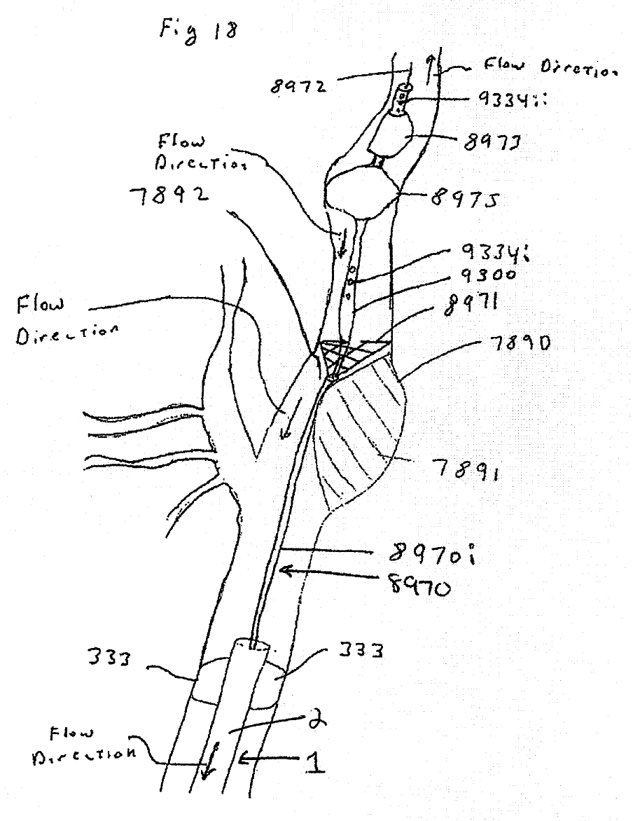

Referring now to FIGS. 15 through 18, it should be noted that the present invention is capable of deployment of multiple supplemental medical (therapeutic) devices (e.g., the delivery catheter 8970 or hypotube 8970i, the fluid delivery (irrigation) catheter (hypotube) 8974, the irrigation catheter 9300, etc.) into a narrowed artery lumen 7892 (FIG. 15) through the operational lumen 2 of the catheter C. For example, as illustrated and described herein, one or more supplemental medical devices may be delivered into the blood vessel V over delivery wire 8972 and either through or over fluid delivery (irrigation) catheter (hypotube) 8974 and/or irrigation catheter 9300. Irrigation catheter 9300 may include optional, multiple fluid-delivery ports 9334, as discussed in connection with the embodiment of the irrigation catheter 9300 seen in FIG. 14. Ports 9334 may be disposed both proximally to temporary occlusion balloon element 8975, and distally to delivered angioplasty balloon 8973. In the embodiment of the disclosure seen in FIG. 18, for example, both balloon elements 8973 and 8975 are mounted on irrigation catheter 9300 distally of stent 8971, with the balloon element 8973 being located distally of the balloon element 8975. Stent element 8971 may also be delivered over the exterior of irrigation catheter 9300 for simultaneous deployment.

It should be further noted that the present invention implements a balloon-guide catheter (or sheath) capable of occluding the target CCA. As indicated in FIG. 18, when the device of the present invention is deployed, fluid flow distal to the temporary occlusion balloon 8975 is static or flows distally, whereas proximally to temporary occlusion balloon 8975 fluid flows proximally through stent 8971, then through tube 1. In a preferred embodiment of the present invention, delivery catheter 8970 (which is configured as hypotube 8970i in the embodiment seen in FIG. 18) is dimensioned sufficiently relatively smaller in diameter versus the inner diameter of tube 1 to allow proximal blood to flow through to tube 1, while having a sufficient inner diameter to deliver over irrigation catheter 9300.

In an alternative embodiment, the described invention relates generally to endovascular devices and more particularly to a specifically using a shaped support catheter and a hypotube in lieu of a wire to shape catheters, as disclosed by the invention. More particularly, the described invention is directed to a device which uses hypotubes, and related elements to obviate the need for open surgical cutdowns of the common carotid artery (CCA) with a carotid stent, using a flow reversal loop system for embolic protection, while also employing a percutaneous technique and novel carotid access devices which use anatomical fulcrums for added support.

Additionally with respect to the embodiments in which one or more of the disclosed devices includes (or is configured as) a hypotube, the present invention combines direct carotid-artery access with rigorous blood flow-reversal, in order to protect the brain from embolic debris when introducing interventional devices into the carotid artery. Disclosed is a medical device capable of treating vascular blockages, more particularly a hypotube having at least one lumen from a proximal port to a distal end hole, capable of delivering additional medical devices; at least one distal, circumferential balloon capable of temporary occlusion of native flow in a vessel near its distal end hole upon inflation. The disclosed hypotube is capable of delivering a second balloon for angioplasty, and at least one stent.

While other Walzman inventions have disclosed the combined use of a wire for curving tubes, and a stent delivery catheter, the present invention discloses a hypotube to perform both of these functions. This configuration eliminates at least one element, thus simplifying the invention, and reducing the possibility of failure. Additionally, by replacing the wire and delivery catheter with a hypotube, the hypotube will be smaller that the combination of those two, thus allowing access to smaller vessels.

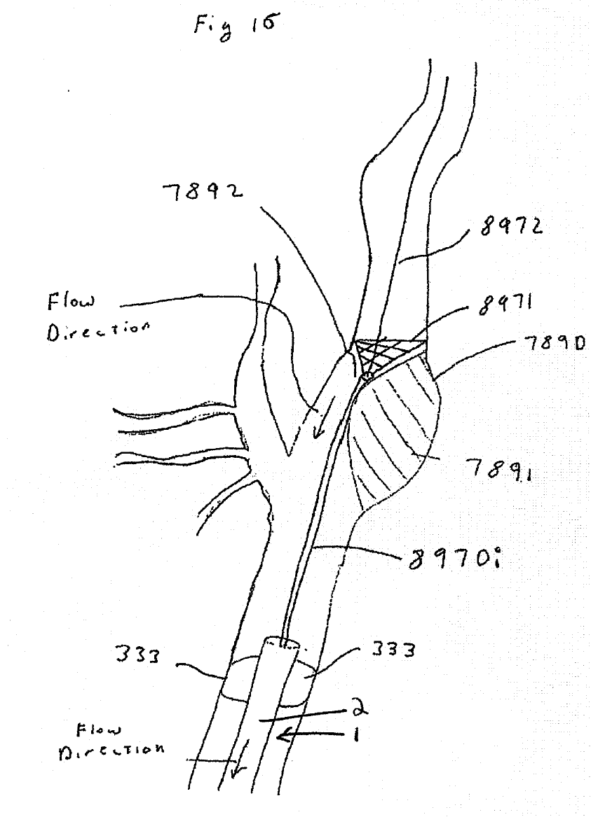

As mentioned above, in certain embodiments of the disclosure, it is envisioned that the delivery catheter 8970 may be configured as (or may be replaced by) hypotube 8970i. For example, in the context of FIG. 16, the narrowed internal carotid-artery lumen 7892 is shown as being accessed by hypotube 8970i, which is configured to deliver stent 8971. FIG. 16 further illustrates (blood) flow direction by arrows. It is envisioned that hypotube 8970i may also be configured to deliver a fluid therethrough to a target area with the vasculature.

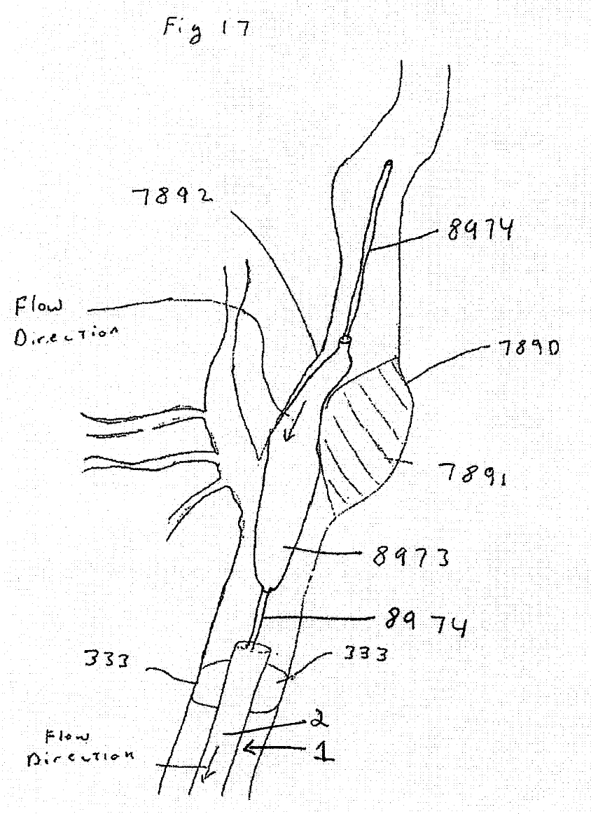

In this alternate embodiment, FIG. 17 illustrates the narrowed internal carotid-artery lumen 7892 of FIG. 15. In the embodiment of the disclosure seen in FIG. 17, angioplasty occlusion balloon 8973 is disposed upon fluid delivery (irrigation) catheter (hypotube) 8974, the direction of blood flow again being shown by arrows. The fluid delivery (irrigation) catheter (hypotube) 8974 is configured for insertion into the vasculature through tube 1.

In this alternate embodiment, FIG. 18 illustrates the narrowed internal carotid-artery lumen 7892 of FIG. 15, further depicting delivery catheter 8970, which is configured as hypotube 8970i, and irrigation catheter 9300. It is envisioned that hypotube 8970i and irrigation catheter 9300 may be connected to each other (e.g., so as to form a single structure). It is also envisioned that hypotube 8970i and irrigation catheter 9300 may be formed as separate, discrete structures (e.g., such that irrigation catheter 9300 is insertable into the blood vessel V through hypotube 8970i).

In the illustrated embodiment, irrigation catheter 9300 is shown as having the aforementioned fluid-delivery ports 9334, which are an optional feature of the structure. More specifically, the first plurality of fluid-delivery ports 9334i are disposed on the irrigation catheter 9300 proximally to temporary occlusion balloon element 8975 and the second plurality of delivery portions 9334ii are disposed on the irrigation catheter 9300 distally to balloon element 8973. As seen in FIG. 18, balloon elements 8973, 8975 are spaced longitudinally (axially) from each other along the length of irrigation catheter 9300; additionally, stent 8971 is also mounted on the exterior of hypotube 8970i. To facilitate delivery of the irrigation catheter 9300 in the manner depicted in FIG. 18, for example, hypotube 8970i includes a diameter allowing balloon elements 8973 and 8975 to pass therethrough. Additionally, as seen in FIG. 18, it is envisioned that stent element 8971 may be mounted on the outer surface of hypotube 8970i. Alternatively, it is envisioned that stent element 8971 may be mounted on the outer surface of the irrigation catheter 9300.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

Any publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of e present invention. All such modifications are intended to be within the scope of the claims appended hereto.

Although the apparatus and methods of the subject invention have been described with respect to preferred embodiments, which constitute non-limiting examples, those skilled in the art will readily appreciate that changes and modifications may be made thereto without departing from the spirit and scope of the present invention as defined by the appended claims.

Additionally, persons skilled in the art will understand that the elements and features shown or described in connection with one embodiment may be combined with those of another embodiment without departing from the scope of the present disclosure and will appreciate further features and advantages of the presently disclosed subject matter based on the description provided.

Throughout the present disclosure, terms such as “approximately,” “generally,” “substantially,” and the like should be understood to allow for variations in any numerical range or concept with which they are associated. For example, it is intended that the use of terms such as “approximately” and “generally” should be understood to encompass variations on the order of 25%, or to allow for manufacturing tolerances and/or deviations in design.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A medical device for treating a vascular narrowing within a blood vessel, the medical device comprising:
a catheter including:
a proximal end hole;
a distal end hole positioned opposite the proximal end hole;
a circumferential balloon located proximally of the distal end hole;
an operational lumen extending through the catheter from the proximal end hole to the distal end hole;
a first bend curving in a first direction; and
a second bend curving in a second direction generally opposite to the first direction, the second bend being positioned distally of the first bend and proximally of the circumferential balloon, wherein the first bend and the second bend are configured to brace the catheter within the blood vessel against an arch defined by the blood vessel to inhibit recoil of the catheter; and
a supplemental medical device configured for insertion into the blood vessel through the operational lumen of the catheter.

2. The medical device of claim 1, wherein the supplemental medical device is configured as a hypotube.

3. The medical device of claim 1, wherein the supplemental medical device is configured as a catheter.

4. The medical device of claim 1, wherein the supplemental medical device supports a stent.

5. The medical device of claim 1, wherein the supplemental medical device includes at least one balloon element.

6. The medical device of claim 5, wherein the supplemental medical device includes a first balloon element and a second balloon element spaced axially from the first balloon element.

7. The medical device of claim 1, wherein the supplemental medical device includes a stent and at least one balloon element.

8. The medical device of claim 7, wherein the at least one balloon element includes a first balloon element located distally of the stent and a second balloon element located distally of the first balloon element.

9. The medical device of claim 8, wherein the supplemental medical device includes a plurality of irrigation ports to facilitate fluid communication through the supplemental medical device into the blood vessel.

10. The medical device of claim 9, wherein the plurality of irrigation ports includes:
a first plurality of irrigation ports located proximally of the first balloon element; and
a second plurality of irrigation ports located distally of the second balloon element.

11. A medical device for treating a vascular narrowing within a blood vessel, the medical device comprising:
a catheter including a tubular body having a first bend curving in a first direction and a second bend curving in a second direction generally opposite to the first direction, the first bend and the second bend being configured to brace the catheter within the blood vessel against an arch defined by the blood vessel to inhibit recoil of the catheter;
a first supplemental medical device configured for insertion into the blood vessel through the catheter; and
a second supplemental medical device configured for insertion into the blood vessel through the first supplemental medical device.

12. The medical device of claim 11, further including a guide wire, the catheter, the first supplemental medical device, and the second supplemental medical device each being configured for insertion into the blood vessel over the guide wire.

13. The medical device of claim 11, wherein the first supplemental medical device includes a stent.

14. The medical device of claim 13, wherein the second supplemental medical device includes at least one balloon element.

15. The medical device of claim 14, wherein the at least one balloon element includes a first balloon element and a second balloon element located distally of the first balloon element, the second supplemental medical device being configured such that the first balloon element and the second balloon element are positionable distally of the stent.

16. The medical device of claim 15, wherein the second supplemental medical device includes a plurality of irrigation ports to facilitate fluid communication through the second supplemental medical device into the blood vessel.

17. The medical device of claim 16, wherein the plurality of irrigation ports includes:
a first plurality of irrigation ports located proximally of the first balloon element; and
a second plurality of irrigation ports located distally of the second balloon element.

18. A medical device for treating a vascular narrowing within a blood vessel, the medical device comprising:
a catheter including:
a tubular body having a plurality of bends curving in a plurality of different directions such that the catheter is configured for bracing against an inner wall of the blood vessel to inhibit recoil of the catheter; and
a circumferential balloon secured to the tubular body;
a first supplemental medical device configured for insertion into the blood vessel through the catheter, the first supplemental medical device including a stent; and
a second supplemental medical device configured for insertion into the blood vessel through the first supplemental medical device, the second supplemental medical device including at least one balloon element.

19. The medical device of claim 18, wherein the second supplemental medical device includes a first plurality of irrigation ports and a second plurality of irrigation ports located distally of the first plurality of irrigation ports, the first plurality of irrigation ports and the second plurality of irrigation ports being configured to facilitate fluid communication through the second supplemental medical device into the blood vessel.

20. The medical device of claim 19, wherein the at least one balloon element is positioned between the first plurality of irrigation ports and the second plurality of irrigation ports.
